(19) United States
(12) Patent Application Publication
(10) Pub. No.: US 2016/0083795 A1
(43) Pub. Date: Mar. 24, 2016

(54) BIOMARKERS

(71) Applicant: NOTTINGHAM TRENT UNIVERSITY, Nottingham, Nottinghamshire (GB)

(72) Inventor: Graham Ball, Nottingham (GB)

(73) Assignee: NOTTINGHAM TRENT UNIVERSITY, Nottingham (GB)

(21) Appl. No.: 14/888,737

(22) PCT Filed: Apr. 30, 2014
(86) PCT No.: PCT/GB2014/051346

§ 371 Date: Nov. 3, 2015

(30) Foreign Application Priority Data
May 3, 2013 (GB) 1308077.5

(51) Int. Cl. C12Q 1/68; G01N 33/68

(52) U.S. Cl. CPC C12Q 1/6883; G01N 33/6896; C12Q 2600/158; C12Q 2600/112; G01N 2800/2821

(57) ABSTRACT

The present invention relates to novel biomarkers for determining the Alzheimer's disease status of a subject, and to uses of novel panels of biomarkers. AD is currently clinically diagnosed using tests of cognitive function. Studies have shown that the accuracy of the clinical diagnosis is between 65 and 90% using cognitive techniques. The present invention provides a method of determining the Alzheimer' s disease status of a subject comprising providing a sample of material obtained from the subject and determining the expression level of one or more genes selected from the group consisting of: TUBB, NEAT1, CENPB, AA482221 and CPSF3 in the sample.

The present invention relates to novel biomarkers for determining the Alzheimer's disease status of a subject, and to uses of novel panels of biomarkers.

The rising global incidence of Alzheimer's disease (AD) poses an increasingly profound problem to society yet progress towards a genuine understanding of the disease remains worryingly slow. As individual life expectancy increases, the incidence of AD worldwide predicted to rise from an estimated 35.6 million people in 2010 to 65.7 million people by 2030.

Perhaps the most outstanding problem with the biology of AD is the question of its mechanistic origins as it remains unclear where the molecular failures occur that underlie the causes and progression of the disease. In other words, changes in the activity or behaviour of (e.g.) specific biochemical pathways which ultimately lead to the cellular damage and neurological disfunction remain unclear. Accordingly, definitions of AD based on the characteristic hallmarks of disease such as amyloid plaques and neurofibrillary tangles associated with brain lesions and cell death represent consequences of the disease rather than the cause.

Currently AD diagnosis is only confirmed post-mortem by the presence of pathological hallmarks including plaques mainly composed of aggregated amyloid-beta (Aβ) and neurofibrillary tangles (NFT) of hyperphosphorylated tau protein. AD is currently clinically diagnosed using tests of cognitive function. Studies have shown that the accuracy of the clinical diagnosis is between 65 and 90% using cognitive techniques. Higher accuracy is achieved at academic centres with special interest in AD. However, this is only at a late stage of the disease when there is extensive irreversible neuronal damage already present.

AD pathology often accumulates over a 10 to 20 year period before clinical symptoms present and even patients with mild cognitive impairment (MCI) have been found to display marked AD pathology at PM.

A reliable, accurate biomarker or set of biomarkers would be an invaluable aid to clinical diagnosis and potentially identify affected individuals much earlier when drug intervention is likely to be most effective. There is an urgent need for reliable and accurate biomarkers to aid clinical diagnosis and progression of Alzheimer's disease (AD).

It would be advantageous to be able to test for the Alzheimer's disease status of an individual subject to distinguish between affected and unaffected individuals, in particular it would be advantageous to be able to test the AD status of an individual at an early stage before symptoms are present or in patients with mild cognitive impairment. Such a test could also be used to follow the AD status of individuals during treatment to determine how the disease is progressing and assess the effect of the treatment.

In a first aspect the present invention provides a method of determining the Alzheimer's disease status of a subject comprising:
(a) providing a sample of material obtained from the subject; and
(b) determining the expression level of one or more genes selected from the group consisting of: TUBB, NEAT1, CENPB, AA482221 and CPSF3 in the sample.

TABLE 1
Control versus AD probes of high significance

| | | |
|---|---|---|
| TUBB | Tubulin Beta Class 1 | Cell structure and |
| | | Transport of proteins |
| | | into and outside of cell |
| NEAT1 | nuclear paraspeckle assembly | Hypothetical role in the |
| | transcript 1 | localisation/ |
| | | compartmentalisation of |
| | | proteins |
| CENPB | Centromere formation | Behaves as a |
| | | microtubule associated |
| | | protein and linked to |
| | | TUBB |
| AA482221 | | Unknown function |
| CPSF3 | Cleavage and polyadenylation | Plays a key role in pre- |
| | specific factor 3 | mRNA 3′-end formation |
| | | brings about cleavage |
| | | and poly(A) addition |

The method may further comprise:
(c) determining the expression level of one or more genes selected from the group consisting of:
   seryl-tRNA synthetase
   stathmin-like 2
   RNA binding motif protein, X-linked
   anaphase promoting complex subunit 13
   reticulon 3
   proteasome (prosome, macropain) subunit, beta type, 3
   hypothetical LOC100509179
   optineurin
   tyrosine 3-monooxygenase/tryptophan 5-monooxygenase
   activation protein, zeta polypeptide
   OCIA domain containing 1
   ATPase, H+ transporting, lysosomal 31 kDa, V1 subunit E1
   fibroblast growth factor (acidic) intracellular binding protein aftiphilin
   Berardinelli-Seip congenital lipodystrophy 2 (seipin)
   glutamic-oxaloacetic transaminase 1, soluble (aspartate aminotransferase 1)
   ATPase, Ca++ transporting, cardiac muscle, slow twitch 2
   DAZ interacting protein 3, zinc finger
   CAP, adenylate cyclase-associated protein 1
   tubulin, alpha 1b
   NADH dehydrogenase (ubiquinone) 1 alpha subcomplex, 7, 14.5 kDa
   eukaryotic translation initiation factor 2B, subunit 3 gamma, 58 kDa
   ribosomal protein L15
   tubulin, alpha 1b
   proteasome (prosome, macropain) 26S subunit, non-ATPase, 1
   proteasome (prosome, macropain) subunit, beta type, 2
   ATP synthase, H+ transporting, mitochondrial F1 complex,
   gamma polypeptide 1
   tetratricopeptide repeat domain 15
   processing of precursor 4, ribonuclease P/MRP subunit (S. cerevisiae)
   rabaptin, RAB GTPase binding effector protein 1.

TABLE 2
Control versus AD probes of significance

| | |
|---|---|
| 1 | seryl-tRNA synthetase |
| 2 | stathmin-like 2 |
| 3 | RNA binding motif protein, X-linked |
| 4 | anaphase promoting complex subunit 13 |
| 5 | reticulon 3 |
| 6 | proteasome (prosome, macropain) subunit, beta type, 3 |
| 7 | hypothetical LOC100509179 |
| 8 | optineurin |
| 9 | tyrosine 3-monooxygenase/tryptophan 5-monooxygenase activation |
| | protein, zeta polypeptide |
| 10 | OCIA domain containing 1 |
| 11 | ATPase, H+ transporting, lysosomal 31 kDa, V1 subunit E1 |
| 12 | fibroblast growth factor (acidic) intracellular binding protein |
| 13 | aftiphilin |
| 14 | Berardinelli-Seip congenital lipodystrophy 2 (seipin) |
| 15 | glutamic-oxaloacetic transaminase 1, soluble (aspartate |
| | aminotransferase 1) |
| 16 | ATPase, Ca++ transporting, cardiac muscle, slow twitch 2 |
| 17 | DAZ interacting protein 3, zinc finger |
| 18 | CAP, adenylate cyclase-associated protein 1 |
| 19 | tubulin, alpha 1b |
| 20 | NADH dehydrogenase (ubiquinone) 1 alpha subcomplex, 7, |
| | 14.5 kDa |
| 21 | eukaryotic translation initiation factor 2B, subunit 3 gamma, |
| | 58 kDa |
| 22 | ribosomal protein L15 |
| 23 | tubulin, alpha 1b |
| 24 | proteasome (prosome, macropain) 26S subunit, non-ATPase, 1 |
| 25 | proteasome (prosome, macropain) subunit, beta type, 2 |
| 26 | ATP synthase, H+ transporting, mitochondrial F1 complex, |
| | gamma polypeptide 1 |
| 27 | tetratricopeptide repeat domain 15 |
| 28 | processing of precursor 4, ribonuclease P/MRP subunit |
| | (S. cerevisiae) |
| 29 | rabaptin, RAB GTPase binding effector protein 1 |

Determination of the level of a biomarker in a sample may comprise the detection of a polypeptide or an mRNA with at least 65% sequence identity, more preferably at least 70%, 75%, 80%, 85%, 90%, 95% or greater sequence identity, to the published sequence of the biomarker, for example the published sequence of a gene listed in Table 1 or Table 2 or the published sequence of a protein encoded by a gene listed in Table 1 or Table 2.

The method may further comprise:
(d) employing the expression level determined in (b) and optionally (c) to distinguish between healthy subjects and subjects having Alzheimer's disease.

The expression level of one, two, three, four or all five genes from Table 1 may be tested in a sample in order to provide an indication that is useful for diagnosing Alzheimer's disease. The more genes that are tested the higher the accuracy of the test.

Where 1 gene selected from Table 1 is used the test may be 84% accurate for predicting Alzheimer's disease in a subject. Where 2 genes selected from Table 1 is used the test may be 86.6% accurate for predicting Alzheimer's disease in a subject. Where 3 genes selected from Table 1 are used the test may be 88.6% accurate for predicting Alzheimer's disease in a subject. Where 4 genes selected from Table 1 are used the test may be 92.8% accurate for predicting Alzheimer's disease in a subject. Where 5 genes selected from Table 1 are used the test may be 94.85% accurate for predicting Alzheimer's disease in a subject.

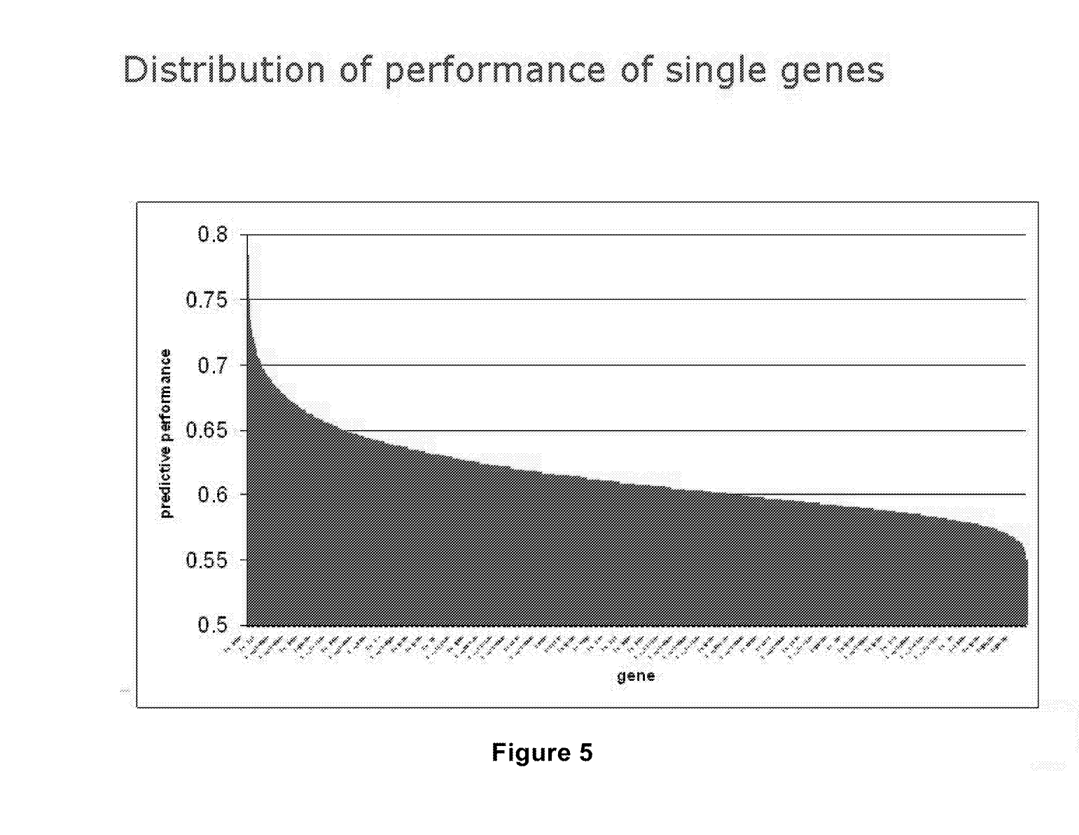

The accuracies of each gene from Table 2 are shown in FIG. 5.

Expression level of any one of the genes from Table 1 and any one or more of the genes in Table 2 may be tested to provide an indication that is useful for diagnosing Alzheimer's disease.

Expression level of two, three, four, or five genes from Table 1 and at least one gene from Table 2 may be tested to provide an indication that is useful in diagnosing Alzheimer's disease.

Expression level of two, three, four, or five genes from Table 1 and at least one gene from Table 2 may be tested to provide an indication that is useful in diagnosing Alzheimer's disease.

The expression level of one or more genes selected from the group in Table 1 and optionally one or more genes selected from Table 2 in a sample may be determined by any suitable method. Such methods include methods that quantify the nucleotide products of these genes such as: quantitative PCR using suitable oligonucleotide primers designed to adhere within the sequence of an mRNA encoded by the gene of interest; analysis of expression arrays; next generation sequencing, comparative genomic hybridisation arrays (CGH arrays); multiplexed PCR. The expression level of one or more genes selected from Table 1 an optionally one or more of the genes selected from Table 2 in a sample may also be determined using methods that quantify the protein products of the selected genes, for example: ELISA; immunohistochemistry; protein aptamer arrays or protein immunological arrays.

The expression level of the gene may be determined by measuring the rate of polymerisation of the RNA using standard techniques.

The method of the invention may not include the step of obtaining the sample.

The subject may be a mammal, and is preferably a human, but may alternatively be a monkey, ape, cat, dog, cow, horse, rabbit or rodent.

The expression level determined in (b) and optionally (c) may be compared to a standard.

The standard may the expression level that would be expected for the same gene in a healthy individual. For example the expression level of a gene in a sample may be compared to the expression level of the same gene in a comparable sample of the same tissue in a healthy individual. The expression level in a sample may be compared to the expected level in a comparable sample from a healthy individual.

The method of the invention may include a further step of comparing the determined value of the expression level of one or more genes selected from Table 1 or Table 2 with a reference value.

The reference value may be the value for the expression level of the same gene in a sample of the same sample type, from an individual who is known to have or not to have Alzheimer's disease. Alternatively, or additionally, the reference value may be the expression level of the same gene in the same sample type in a sample taken previously from the same subject, for example, prior to or during the course of a particular treatment. The reference sample may be a sample of the same type, for example, both samples may be blood samples. In this way the expression level of one or more genes selected from Table 1 or Table 2 may be used to monitor the progression of disease in a subject, and/or to monitor the efficacy of a particular treatment in a subject.

The method of the invention may be carried out in vitro.

About a 10% or more increase in expression of a biomarker compared to the level in a normal sample may be diagnostic of Alzheimer's disease.

About a 20% or more increase in expression of a biomarker compared to the level in a normal sample may be diagnostic of Alzheimer's disease.

About a 30% or more increase in expression of a biomarker compared to the level in a normal sample may be diagnostic of Alzheimer's disease.

About a 40% or more increase in expression of a biomarker compared to the level in a normal sample may be diagnostic of Alzheimer's disease.

About a 50% or more increase in expression of a biomarker compared to the level in a normal sample may be diagnostic of Alzheimer's disease.

About a 60% or more increase in expression of a biomarker compared to the level in a normal sample may be diagnostic of Alzheimer's disease.

About a 70% or more increase in expression of a biomarker compared to the level in a normal sample may be diagnostic of Alzheimer's disease.

About a 80% or more increase in expression of a biomarker compared to the level in a normal sample may be diagnostic of Alzheimer's disease.

About a 90% or more increase in expression of a biomarker compared to the level in a normal sample may be diagnostic of Alzheimer's disease.

About a 100% or more increase in expression of a biomarker compared to the level in a normal sample may be diagnostic of Alzheimer's disease.

About a 10% or more decrease in expression of a biomarker compared to the level in a normal sample may be diagnostic of Alzheimer's disease.

About a 20% or more decrease in expression of a biomarker compared to the level in a normal sample may be diagnostic of Alzheimer's disease.

About a 30% or more decrease in expression of a biomarker compared to the level in a normal sample may be diagnostic of Alzheimer's disease.

About a 40% or more decrease in expression of a biomarker compared to the level in a normal sample may be diagnostic of Alzheimer's disease.

About a 50% or more decrease in expression of a biomarker compared to the level in a normal sample may be diagnostic of Alzheimer's disease.

About a 60% or more decrease in expression of a biomarker compared to the level in a normal sample may be diagnostic of Alzheimer's disease.

About a 70% or more decrease in expression of a biomarker compared to the level in a normal sample may be diagnostic of Alzheimer's disease.

About a 80% or more decrease in expression of a biomarker compared to the level in a normal sample may be diagnostic of Alzheimer's disease.

About a 90% or more decrease in expression of a biomarker compared to the level in a normal sample may be diagnostic of Alzheimer's disease.

About a 100% or more decrease in expression of a biomarker compared to the level in a normal sample may be diagnostic of Alzheimer's disease.

In a panel of biomarkers an increase in expression of some biomarkers compared to the level in a normal sample and a decrease in other biomarkers compared to the level in a normal sample may be diagnostic of Alzheimer's disease.

A panel of biomarkers may comprise or consist of TUBB, NEAT1, CENPB, AA482221 and CPSF3. A Decrease in expression of TUBB and an increase in the expression of NEAT1 and an increase in the expression of CENPB and a decrease in the expression of AA482221 and a decrease in expression of CPSF3 may be diagnostic of Alzheimer's disease. This panel of biomarkers may have about 94% or about 94.85% accuracy in predicting Alzheimer's disease.

A panel of biomarkers may comprise or consist of TUBB, NEAT1, CENPB and AA482221. A Decrease in expression of TUBB and an increase in the expression of NEAT1 and an increase in the expression of CENPB and a decrease in the expression of AA482221 may be diagnostic of Alzheimer's disease. This panel of biomarkers may have about 92% or about 92.78% accuracy in predicting Alzheimer's disease.

A panel of biomarkers may comprise or consist of TUBB, NEAT1 and CENPB. A Decrease in expression of TUBB and an increase in the expression of NEAT1 and an increase in the expression of CENPB may be diagnostic of Alzheimer's disease. This panel of biomarkers may have about 88% or about 88.66% accuracy in predicting Alzheimer's disease.

A panel of biomarkers may comprise or consist of TUBB and NEAT1. A Decrease in expression of TUBB and an increase in the expression of NEAT1 may be diagnostic of Alzheimer's disease. This panel of biomarkers may have about 86% or about 86.60% accuracy in predicting Alzheimer's disease.

A panel of biomarkers may comprise or consist of TUBB. A Decrease in expression of TUBB may be diagnostic of Alzheimer's disease. This panel of biomarkers may have about 84% or 84.02% accuracy in predicting Alzheimer's disease.

A panel of biomarkers may comprise or consist of TUBB, NEAT1, CENPB, AA482221 and CPSF3 and one additional biomarker selected from Table 2.

A panel of biomarkers may comprise or consist of TUBB, NEAT1, CENPB, AA482221 and CPSF3 and two additional biomarkers selected from Table 2.

A panel of biomarkers may comprise or consist of TUBB, NEAT1, CENPB, AA482221 and CPSF3 and three additional biomarkers selected from Table 2.

A panel of biomarkers may comprise or consist of TUBB, NEAT1, CENPB, AA482221 and CPSF3 and four additional biomarkers selected from Table 2.

A panel of biomarkers may comprise or consist of TUBB, NEAT1, CENPB, AA482221 and CPSF3 and five additional biomarkers selected from Table 2.

A biomarker panel may be 6 biomarkers giving an accuracy of about 95% in predicting Alzheimer's disease.

A biomarker panel may comprise or consist of TUBB and one additional biomarker selected from Table 2.

A biomarker panel may comprise or consist of TUBB and two additional biomarkers selected from Table 2.

A biomarker panel may comprise or consist of TUBB and three additional biomarkers selected from Table 2.

A biomarker panel may comprise or consist of TUBB and four additional biomarkers selected from Table 2.

A biomarker panel may comprise or consist of TUBB and five additional biomarkers selected from Table 2. This biomarker panel may provide about 95% accuracy in predicting Alzheimer's disease.

Use of a biomarker panel comprising TUBB as a biomarker for Alzheimer's disease. Wherein expression of TUBB is decreased in a sample from a subject with Alzheimer's disease compared to a comparable sample from a healthy individual.

Use of a biomarker panel comprising NEAT1 as a biomarker for Alzheimer's disease. Wherein expression of NEAT1 is decreased in a sample from a subject with Alzheimer's disease compared to a comparable sample from a healthy individual.

Use of a biomarker panel comprising CENPB as a biomarker for Alzheimer's disease. Wherein expression of CENPB is decreased in a sample from a subject with Alzheimer's disease compared to a comparable sample from a healthy individual.

Use of a biomarker panel comprising AA482221 as a biomarker for Alzheimer's disease. Wherein expression of AA482221 is decreased in a sample from a subject with Alzheimer's disease compared to a comparable sample from a healthy individual.

Use of a biomarker panel comprising CPSF3 as a biomarker for Alzheimer's disease. Wherein expression of CPSF3 is decreased in a sample from a subject with Alzheimer's disease compared to a comparable sample from a healthy individual.

Use of a biomarker panel comprising seryl-tRNA synthetase for detection of Alzheimer's disease.

Use of a biomarker panel comprising stathmin-like 2 for detection of Alzheimer's disease.

Use of a biomarker panel comprising RNA binding motif protein, X-linked for detection of Alzheimer's disease.

Use of a biomarker panel comprising anaphase promoting complex subunit 13 for detection of Alzheimer's disease.

Use of a biomarker panel comprising reticulon 3 for detection of Alzheimer's disease.

Use of a biomarker panel comprising proteasome (prosome, macropain) subunit, beta type, 3 for detection of Alzheimer's disease.

Use of a biomarker panel comprising hypothetical LOC100509179 for detection of Alzheimer's disease.

Use of a biomarker panel comprising optineurin for detection of Alzheimer's disease.

Use of a biomarker panel comprising tyrosine 3-monooxygenase/tryptophan 5-monooxygenase activation protein, for detection of Alzheimer's disease.

Use of a biomarker panel comprising zeta polypeptide for detection of Alzheimer's disease.

Use of a biomarker panel comprising OCIA domain containing 1 for detection of Alzheimer's disease.

Use of a biomarker panel comprising ATPase, H+ transporting, lysosomal 31 kDa, V1 subunit E1 for detection of Alzheimer's disease.

Use of a biomarker panel comprising fibroblast growth factor (acidic) intracellular binding protein for detection of Alzheimer's disease.

Use of a biomarker panel comprising aftiphilin for detection of Alzheimer's disease.

Use of a biomarker panel comprising Berardinelli-Seip congenital lipodystrophy 2 (seipin) for detection of Alzheimer's disease.

Use of a biomarker panel comprising glutamic-oxaloacetic transaminase 1, soluble (aspartate aminotransferase 1) for detection of Alzheimer's disease.

Use of a biomarker panel comprising ATPase, Ca++ transporting, cardiac muscle, slow twitch 2 for detection of Alzheimer's disease.

Use of a biomarker panel comprising DAZ interacting protein 3, zinc finger for detection of Alzheimer's disease.

Use of a biomarker panel comprising CAP, adenylate cyclase-associated protein 1 for detection of Alzheimer's disease.

Use of a biomarker panel comprising tubulin, alpha 1b for detection of Alzheimer's disease.

Use of a biomarker panel comprising NADH dehydrogenase (ubiquinone) 1 alpha subcomplex, 7, 14.5 kDa for detection of Alzheimer's disease.

Use of a biomarker panel comprising eukaryotic translation initiation factor 2B, subunit 3 gamma, 58 kDa for detection of Alzheimer's disease.

Use of a biomarker panel comprising Use of a biomarker panel comprising ribosomal protein L15 for detection of Alzheimer's disease.

Use of a biomarker panel comprising tubulin, alpha 1b for detection of Alzheimer's disease.

Use of a biomarker panel comprising proteasome (prosome, macropain) 26S subunit, non-ATPase, 1 for detection of Alzheimer's disease.

Use of a biomarker panel comprising proteasome (prosome, macropain) subunit, beta type, 2 for detection of Alzheimer's disease.

Use of a biomarker panel comprising ATP synthase, H+ transporting, mitochondrial F1 complex, gamma polypeptide 1 for detection of Alzheimer's disease.

Use of a biomarker panel comprising tetratricopeptide repeat domain 15 for detection of Alzheimer's disease.

Use of a biomarker panel comprising processing of precursor 4, ribonuclease P/MRP subunit (S. cerevisiae) for detection of Alzheimer's disease.

Use of a biomarker panel comprising rabaptin, RAB GTPase binding effector protein 1 for detection of Alzheimer's disease.

Where a sample from a subject shows only a slight increase or a decrease in the amount of one or more biomarkers the subject may be classified as having mild cognitive impairment (MCI) rather than Alzheimer's disease.

The biomarkers of the invention may be used as one or more of diagnostic biomarkers, prognostic biomarkers and therapeutic biomarkers. Biomarkers which are not explicitly stated to be therapeutic biomarkers are preferably at least one of diagnostic or prognostic biomarkers, or both.

Preferably the method allows the diagnosis of Alzheimer's disease in a subject from the analysis of the level of the one or more biomarkers in a sample provided by/or obtained from the subject.

The method of the invention may also or alternatively be used to develop therapies targeted to the biomarkers. Reference herein to therapeutic biomarkers is intended to refer to biomarkers which can be used as targets for therapy, this may be in addition to or an alternative to the biomarkers being used for diagnostic and/or prognostic purposes. Preferably a therapeutic biomarker represents a protein which can be targeted by a ligand to deliver a drug, for example, the therapeutic biomarker may be recognised by an antibody-drug conjugate wherein the antibody recognises the therapeutic biomarker and delivers the conjugated drug.

If the expression level of one or more of the genes selected from Table 1 and optionally Table 2 is elevated or decreased relative to the standard this indicates that the subject may have Alzheimer's disease.

If the expression level of one or more genes from Table 1 and optionally Table 2 in a sample from an individual is found to be higher or lower than expected this may indicate that the indicate that the individual may have Alzheimer's disease. The individual may be selected for treatment for Alzheimer's disease and the individual may be treated for Alzheimer's disease.

The method may be used in one or more of the following; diagnosing whether or not a subject has Alzheimer's disease; advising on the prognosis for a subject with Alzheimer's disease and monitoring the effectiveness or response of a subject to a particular treatment for Alzheimer's disease.

At least one of the genes from Table 1 and optionally at least one of the genes from Table 2 may be used as a biomarker panel where the expression level of genes is measured to distinguish between subjects having Alzheimer's disease and healthy subjects with a high degree of accuracy. The accuracy is increased as the number of biomarkers increases. This biomarker panel is advantageous because it provides a high degree of accuracy with only a small number of biomarkers.

The altered expression level is an expression level that is higher or lower than the expression level expected in a subject not having Alzheimer's disease.

The expression level of the gene may be determined using the level of mRNA encoded by the selected gene present in the sample.

The expression level of the gene may be determined using quantitative PCR.

The expression level of the gene may be determined using the level of protein encoded by the selected gene present in the sample.

The expression level of one or more further genes may also be determined.

In another aspect the present invention provides a kit for use in determining the Alzheimer's disease status in a subject comprising at least one agent for determining the expression level of one or more genes selected from the group consisting of: TUBB, NEAT1, CENPB, AA482221 and CPSF3 in a sample from a subject and instructions for determining the Alzheimer's disease status of the subject.

The kit may further comprise at least one agent for determining the expression level of one or more genes listed in Table 2.

The agent may be an oligonucleotide.

In a further aspect the present invention provides use of the determination of the expression level of one or more genes selected from the group consisting of: TUBB, NEAT1, CENPB, AA482221 and CPSF3 and optionally one or more genes from Table 2 as a means to determine the

The present invention further provides a gene expression product from a gene selected from the group consisting of TUBB, NEAT1, CENPB, AA482221 and CPSF3 or the expression product from a gene listed in Table 2 for use as biomarker for subjects having Alzheimer's disease.

The present invention further provides an oligonucleotide capable of detecting the presence or expression level of a gene expression product from a gene selected from the group consisting of TUBB, NEAT1, CENPB, AA482221 and CPSF3 or a gene selected from the genes listed in Table 2 in a sample from a subject.

The present invention further provides a method, kit, use, gene or oligonucleotide as described herein wherein the sample is a sample of blood, serum, cerebrospinal fluid, saliva, urine, tissue fluid, tissue, brain tissue.

The sample of material may be a sample of blood, sputum, saliva, wound exudate, urine, faeces, peritoneal fluid or any respiratory secretion. A sample of blood may be whole blood, blood plasma or blood serum.

The sample may be a sample of whole blood or a fraction of whole blood. Blood samples have the advantage that they are readily obtainable and tend to be more homogenous in nature than other sample types. Samples of whole blood contain RNA which can be extracted to generate a transcriptional profile.

The sample may be a sample of cerebrospinal fluid.

Where the sample is a sample of blood, whole blood, a fraction of whole blood or a sample of cerebrospinal fluid the biomarkers may be protein products of the genes listed in Table 1 or Table 2.

The methods of the present invention are advantageous as they permit both the regular screening of susceptible populations and also the testing of all individuals who are suspected of having Alzheimer's disease, and thus enables more timely treatment possibly preventing or reducing the impact of symptoms. It also permits earlier diagnosis than is available using conventional diagnosis techniques and therefore Alzheimer's disease can be treated earlier. It also permits a quantitative assessment of the efficacy of therapy resulting in interventions that are customised to the response of the individual patient. It also provides a tool for widespread use in epidemiological studies, an important consideration as Alzheimer's disease becomes more prevalent.

The genes listed in Table 1 and Table 2 may be targets for development of new therapeutics for treating Alzheimer's disease.

The skilled man will appreciate that preferred features of any one embodiment and/or aspect of the invention may be applied to all other embodiments or aspects of the invention.

The present invention will be further described in more detail, by way of example only, with reference to the following Figures in which:

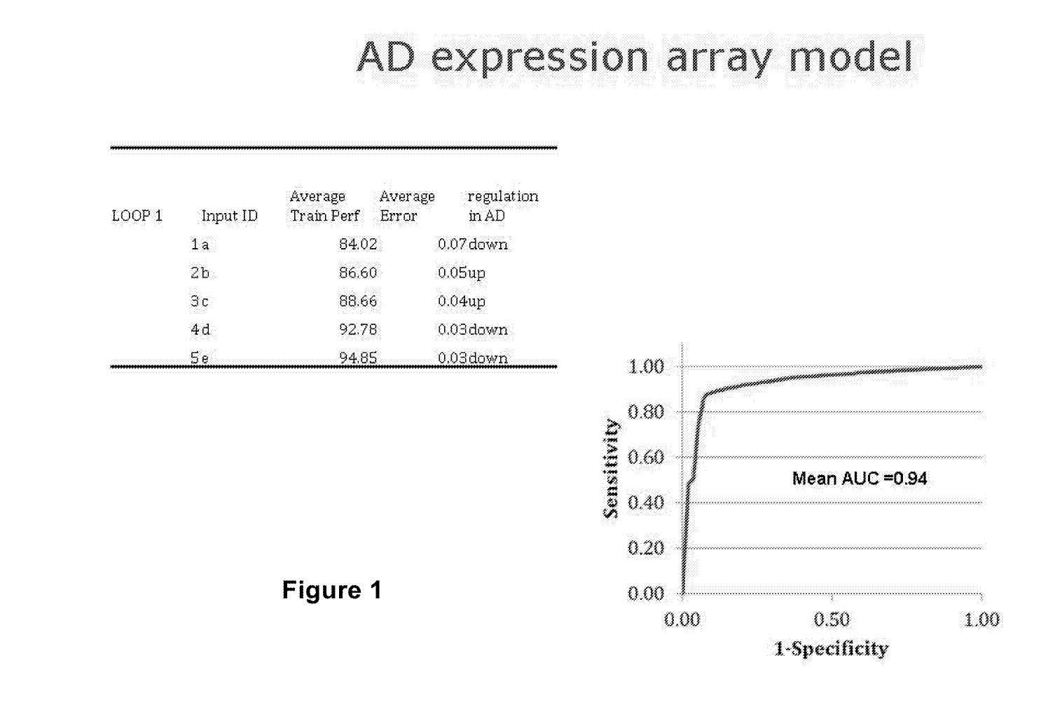

FIG. 1 shows data from an Alzheimer's disease expression array model;

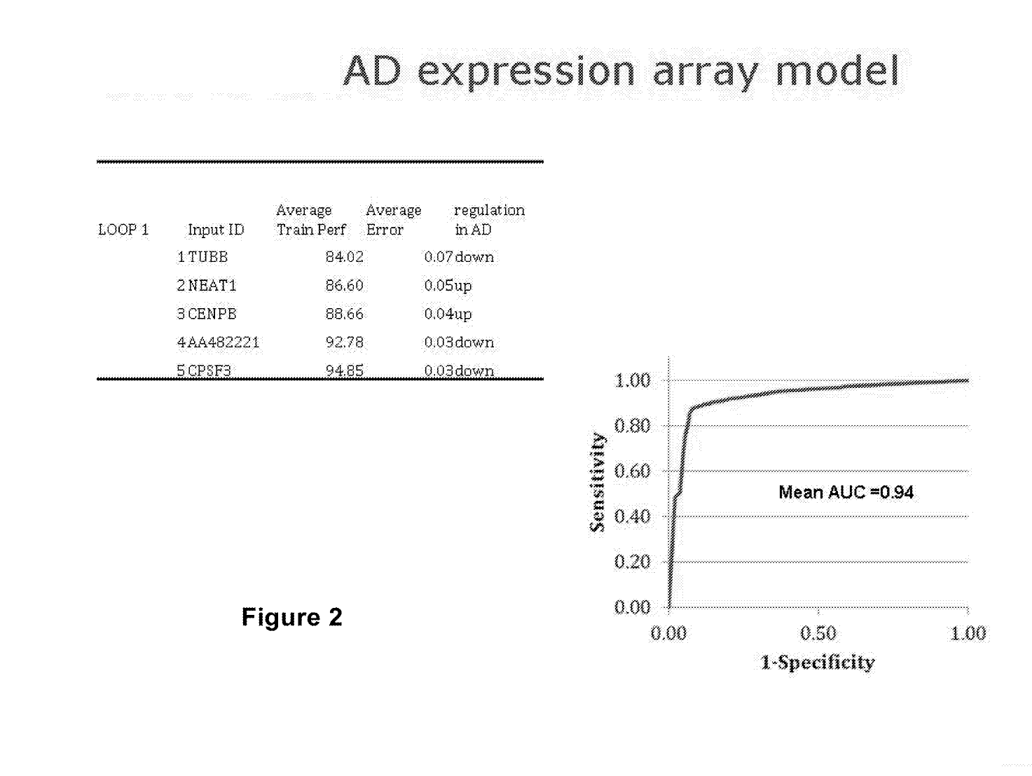

FIG. 2 shows data from an Alzheimer's disease expression array model;

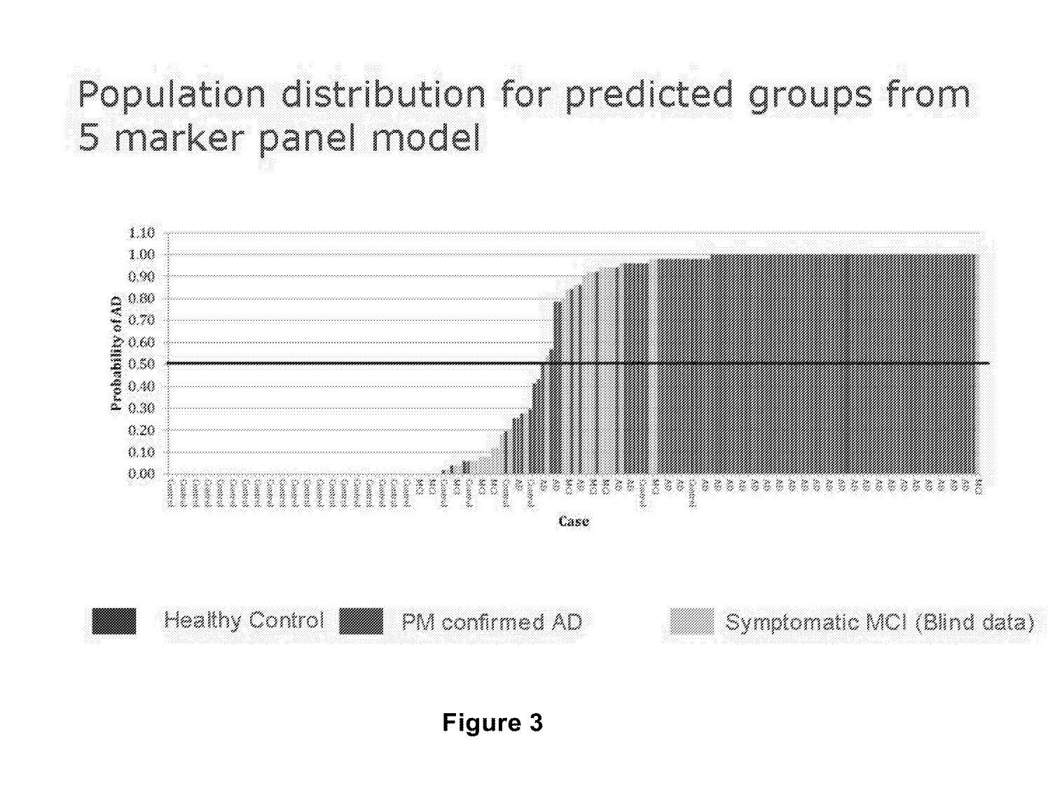

FIG. 3 shows the population distribution for predicted groups from 5 marker panel model;

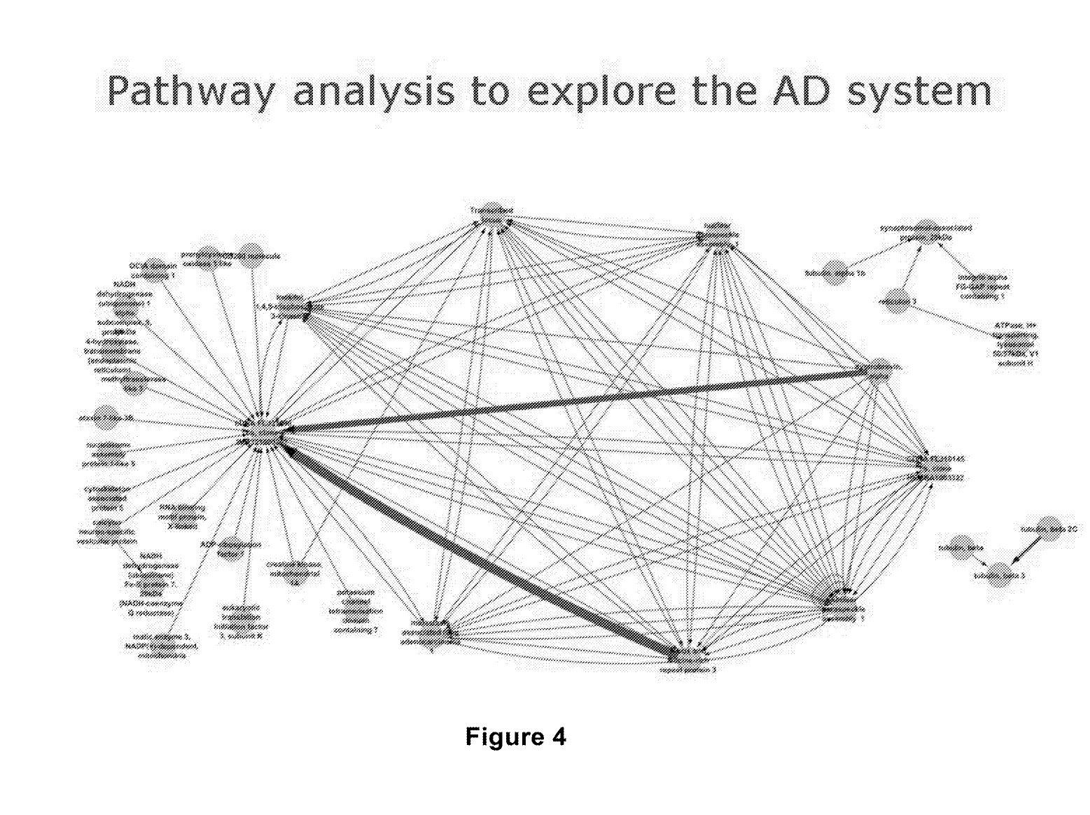

FIG. 4 shows a pathway analysis to explore the Alzheimer's disease system; and

FIG. 5 shows the distribution of performance of single genes as biomarkers for predicting Alzheimer's disease.

Raw data used to produce the gene panels was taken from Liang et al., Physiol Genomics, 2007, Feb. 12; 28(3): 311-322.

In Liang et al data was obtained from Tissue samples (200 μm of sectioned tissue) from brain regions histopathologically or metabolically relevant to AD. Gene expression profiles from laser capture microdissected neurons in six functionally and anatomically distinct regions from clinically and histopathologically normal aged human brains were produced. These regions, which are also known to be differentially vulnerable to the histopathological and metabolic features of Alzheimer's disease (AD), they include the entorhinal cortex and hippocampus (limbic and paralimbic areas vulnerable to early neurofibrillary tangle pathology in AD), posterior cingulate cortex (a paralimbic area vulnerable to early metabolic abnormalities in AD), temporal and prefrontal cortex (unimodal and heteromodal sensory association areas vulnerable to early neuritic plaque pathology in AD), and primary visual cortex (a primary sensory area relatively spared in early AD). These neuronal profiles will provide valuable reference information for future studies of the brain, in normal aging, AD and other neurological and psychiatric disorders.

Presence of AD was confirmed at post mortem for each of the brains that AD tissue was obtained from.

Plaque regions were histologically identified for each brain and isolated by LCM.

Profiled using Affymetrix U133 Plus 2.0 array

N=89 AD, 74 controls (age matched), 34 MCI—BY MMSE (blind samples)

The data was analysed using a data mining algorithm and method both described in patent application number PCT/GB2009/051412, published as WO2010/046697 and claiming priority to GB 0819221.3. This method provided two panels of biomarkers.
1. Table 1 is a panel of biomarkers which were used together in a diagnostic model. All 5 of these markers were used together and separately to predict whether a subject has Alzheimer's disease. The accuracy of the prediction was calculated and the results shown in FIG. 2. In FIG. 2 it is shown whether each biomarker is up regulated or down regulated in samples from subjects with Alzheimer's disease compared to samples from healthy subjects (right hand column of FIG. 3). The cumulative percentage accuracy is shown in the 3^{rd} column in the Table in FIG. 2, for example, TUBB is down regulated in samples from subjects with Alzheimer's disease compared to samples from healthy subjects. The accuracy of prediction of Alzheimer's disease with TUBB alone was calculated as 84.02%. The Accuracy of prediction of Alzheimer's disease increased with each additional biomarker as shown in the Table in FIG. 2 where the percentage accuracy is shown in the column marked “Average Train Perf” and the percentage accuracy is cumulative going down that column.
2. The panel of biomarkers in Table 2 are single markers which are predictive on their own but may constitute an improved signature or alternative solution when added to those in Table 1.

1. A method of determining the Alzheimer's disease status of a subject comprising:
(a) providing a sample of material obtained from the subject; and
(b) determining the expression level of one or more genes selected from the group consisting of: TUBB, NEAT1, CENPB, AA482221 and CPSF3 in the sample.

2. The method of claim 1 further comprising:
(c) determining the expression level of one or more genes selected from the group consisting of:
tubulin, alpha 1c
seryl-tRNA synthetase
stathmin-like 2
RNA binding motif protein, X-linked
anaphase promoting complex subunit 13
reticulon 3
proteasome (prosome, macropain) subunit, beta type, 3
hypothetical LOC100509179
optineurin
tyrosine 3-monooxygenase/tryptophan 5-monooxygenase activation
protein, zeta polypeptide
OCIA domain containing 1
ATPase, H+ transporting, lysosomal 31 kDa, V1 subunit E1
fibroblast growth factor (acidic) intracellular binding protein
aftiphilin
Berardinelli-Seip congenital lipodystrophy 2 (seipin)
glutamic-oxaloacetic transaminase 1, soluble (aspartate aminotransferase 1)
ATPase, Ca++ transporting, cardiac muscle, slow twitch 2
DAZ interacting protein 3, zinc finger
CAP, adenylate cyclase-associated protein 1
tubulin, alpha 1b
NADH dehydrogenase (ubiquinone) 1 alpha subcomplex, 7, 14.5 kDa
eukaryotic translation initiation factor 2B, subunit 3 gamma, 58 kDa
ribosomal protein L15
tubulin, alpha 1b
proteasome (prosome, macropain) 26S subunit, non-ATPase, 1
proteasome (prosome, macropain) subunit, beta type, 2
ATP synthase, H+ transporting, mitochondrial F1 complex, gamma
polypeptide 1
tetratricopeptide repeat domain 15
processing of precursor 4, ribonuclease P/MRP subunit (S. cerevisiae)
rabaptin, RAB GTPase binding effector protein 1.

3. The method of claim 1 further comprising:
(d) employing the expression level determined in (b) and optionally (c) to distinguish between healthy subjects and subjects having Alzheimer's disease.

4. The method of claim 3 wherein the expression level determined in (b) and optionally (c) is compared to a standard.

5. The method of claim 4 wherein the standard is the expression level that would be expected in a healthy individual.

6. The method according to claim 4 wherein if the expression level of one or more of the genes is elevated or decreased relative to the standard this indicates that the subject may have Alzheimer's disease.

7. The method of claim 4 wherein a subject having Alzheimer's disease is selected for treatment.

8. The method of claim 4 wherein a subject having Alzheimer's disease is treated for Alzheimer's disease.

9. The method of claim 1 for diagnosing whether or not a subject has Alzheimer's disease; advising on the prognosis for a subject with Alzheimer's disease and monitoring the effectiveness or response of a subject to a particular treatment for Alzheimer's disease.

10. The method of claim 1 wherein altered expression level of one, two, three, four or five genes selected from: TUBB, NEAT1, CENPB, AA482221 and CPSF3 can differentiate between healthy subjects and subjects having Alzheimer's disease.

11. The method of claim 1 wherein the altered expression level is an expression level that is higher or lower than the expression level expected in a subject not having Alzheimer's disease.

12. A method of claim 1 wherein the expression level of the gene is determined using the level of mRNA encoded by the selected gene present in the sample.

13. A method according claim 12 wherein the expression level of the gene is determined using quantitative PCR.

14. A method of claim 1 wherein the expression level of the gene is determined using the level of protein encoded by the selected gene present in the sample.

15. A method of claim 1 wherein the expression level of one or more further genes is also determined.

16. A kit for use in determining the Alzheimer's disease status in a subject comprising at least one agent for determining the expression level of one or more genes selected from the group consisting of: TUBB, NEAT1, CENPB, AA482221 and CPSF3 in a sample from a subject and instructions for determining the Alzheimer's disease status of the subject.

17. A kit according to claim 16 further comprising at least one agent for determining the expression level of one or more of the following genes:
tubulin, alpha 1c
seryl-tRNA synthetase
stathmin-like 2
RNA binding motif protein, X-linked
anaphase promoting complex subunit 13
reticulon 3
proteasome (prosome, macropain) subunit, beta type, 3
hypothetical LOC100509179
optineurin
tyrosine 3-monooxygenase/tryptophan 5-monooxygenase activation
protein, zeta polypeptide
OCIA domain containing 1
ATPase, H+ transporting, lysosomal 31 kDa, V1 subunit E1
fibroblast growth factor (acidic) intracellular binding protein
aftiphilin
Berardinelli-Seip congenital lipodystrophy 2 (seipin)
glutamic-oxaloacetic transaminase 1, soluble (aspartate
aminotransferase 1)
ATPase, Ca++ transporting, cardiac muscle, slow twitch 2
DAZ interacting protein 3, zinc finger
CAP, adenylate cyclase-associated protein 1
tubulin, alpha 1b
NADH dehydrogenase (ubiquinone) 1 alpha subcomplex, 7, 14.5 kDa
eukaryotic translation initiation factor 2B, subunit 3 gamma, 58 kDa
ribosomal protein L15
tubulin, alpha 1b
proteasome (prosome, macropain) 26S subunit, non-ATPase, 1
proteasome (prosome, macropain) subunit, beta type, 2
ATP synthase, H+ transporting, mitochondrial F1 complex, gamma
polypeptide 1
tetratricopeptide repeat domain 15
processing of precursor 4, ribonuclease P/MRP subunit (S. cerevisiae) rabaptin,
RAB GTPase binding effector protein 1.

18. The kit of claim 16 wherein the agent is an oligonucleotide.

19-24. (canceled)

25. A method of selecting a subject for treatment for Alzheimer's disease comprising the steps of:
i) determining amount of one or more biomarkers selected from, TUBB, NEAT1, CENPB, AA482221 and CPSF3, and optionally one or more of the biomarkers listed in claim 2, in a sample from the subject;
ii) determining if the amount of each of the one or more biomarkers is elevated or decreased relative to the expected level of that biomarker in a comparable sample from a healthy subject,
wherein the subject is selected for treatment for Alzheimer's disease or for further investigation of Alzheimer's disease if the subject is determined to have elevated or reduced amounts of the one or more biomarkers.

26. The method according to claim 25 further comprising the step of administering a drug suitable for treatment of Alzheimer's disease to the subject.

27. (canceled)
